(19) United States
(12) Patent Application Publication
(10) Pub. No.: US 2023/0346665 A1
(43) Pub. Date: Nov. 2, 2023

(54) ANHYDROUS COSMETIC COMPOSITION COMPRISING POLYOLS, MONOALCOHOLS, FATTY AMINES AND AT LEAST 0.1% OF CARBOXYLIC ACIDS

(71) Applicant: L'OREAL, PARIS (FR)

(72) Inventor: Saber MALOUG, SAINT-OUEN (FR)

(21) Appl. No.: 17/921,799

(22) PCT Filed: Apr. 26, 2021
(86) PCT No.: PCT/EP2021/060834

§ 371 Date: Oct. 27, 2022

(30) Foreign Application Priority Data
Apr. 30, 2020 (FR) FR2004289

(51) Int. Cl. A61K 8/42; A61K 8/365; A61K 8/37; A61K 8/34; A61Q 5/12

(52) U.S. Cl. CPC A61K 8/42; A61K 8/365; A61K 8/37; A61K 8/342; A61Q 5/12; A61K 8/345; A61K 2800/31; A61K 2800/262

(57) ABSTRACT

The present invention relates to an anhydrous cosmetic composition comprising: (i) one or more polyols, (ii) one or more monoalcohols comprising 2 to 6 carbon atoms, (iii) one or more fatty amines, and (iv) at least 0.1% by weight, relative to the total weight of said composition, of one or more carboxylic acids comprising from 1 to 6 carbon atoms. The invention also relates to a cosmetic process for treating keratin materials such as the hair, comprising the application to said keratin materials of such a composition.

The invention relates to a cosmetic composition comprising one or more monoalcohols and one or more polyols, and also one or more fatty amines and one or more carboxylic acids; the invention also relates to a cosmetic process for treating keratin fibers, in particular human keratin fibers such as the hair, using said cosmetic composition.

Hair is generally damaged and embrittled by the action of external atmospheric agents such as light and bad weather, and also by mechanical or chemical treatments, such as brushing, combing, dyeing, bleaching, permanent-waving and/or relaxing, or even repeated washing.

Hair is thus damaged by these various factors and may over time become dry, coarse, brittle or dull, notably in fragile areas, and more particularly at the ends.

To overcome these drawbacks, it is common practice to resort to haircare treatments using compositions that condition the hair, giving it satisfactory cosmetic properties, notably in terms of smoothness, sheen, softness, suppleness, lightness, a natural feel and good disentangling properties. These haircare compositions intended to be applied regularly to the hair may be, for example, hair conditioners, masks or sera, and may be in the form of gels, hair lotions or care creams that are more or less thick.

The products present on the market generally have a thick texture and are difficult to apply and to spread over the entire head of hair. The user thus finds these products rather unpleasant to use.

In addition, users are increasingly in search of products mainly comprising ingredients of natural origin.

The aim of the present invention is to propose hair compositions that are easy to use, notably easy to apply to the hair, and which provide keratin fibers with good properties, notably sensory and care properties, while at the same time mainly comprising ingredients of natural origin.

Preferably, the compositions according to the invention do not comprise (0%) any surfactants bearing a quaternary ammonium group, such as dimethyldialkylammonium or trimethylalkylammonium compounds, having alkyl chain lengths of from C8 to C22, for example.

Preferably also, the compositions according to the invention do not comprise (0%) any cationic polymers, notably bearing vinyl units.

One subject of the invention is thus an anhydrous cosmetic composition comprising:

(i) one or more polyols,

(ii) one or more monoalcohols comprising 2 to 6 carbon atoms,

(iii) one or more fatty amines, and

(iv) at least 0.1% by weight, relative to the total weight of said composition, of one or more carboxylic acids comprising from 1 to 6 carbon atoms.

A subject of the invention is also a cosmetic process for treating keratin materials, in particular human keratin fibers such as the hair, comprising the application to said keratin materials of a composition as defined above.

The composition according to the invention is advantageously in the form of a transparent composition, notably having a transmittance measurement of at least 80%, better still of at least 90%, even better still of at least 95%, measured at a wavelength of 600 nm, for example using a Perkin-Elmer Lambda 40 UV-VIS spectrometer.

The composition according to the invention has good working qualities, and in particular an adequate viscosity which enables it to be applied and to be spread easily over the head of hair.

In particular, the composition according to the invention has a viscosity which may range from 10 mPa·s to 10 000 mPa·s at 25° C. The viscosity may be measured with a Brookfield RVT viscometer at 20 rpm (revolutions/minute), at 25° C., the spindle size being selected according to the manufacturer's standard recommendations, generally ranging from the disk spindle No. 1 to No. 4.

In addition, the composition according to the invention gives keratin fibers good cosmetic properties, such as a homogeneous soft, smooth feel and also suppleness. The keratin fibers are also easy to disentangle and are not made heavy by the composition.

Other subjects, features, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

The expression “at least one” used in the present description is equivalent to the expression “one or more”.

The composition according to the invention is anhydrous.

The term “anhydrous” means that the composition comprises not more than 5% by weight of water, relative to the total weight of the final composition, preferably not more than 2% water, better still not more than 1% water, even better still not more than 0.5% water, and is preferentially free of water (0%).

### 1/ Polyols

The composition according to the invention comprises one or more polyols.

Preferably, said polyol(s) are linear and saturated; they may comprise from 2 to 6 hydroxyl OH groups, notably from 2 to 4, better still from 2 to 3 and preferentially 2 OH groups.

They may comprise from 2 to 8 carbon atoms and notably from 2 to 6 carbon atoms.

They do not comprise any oxyalkylene or glycerol-based groups. They are advantageously liquid at room temperature (25° C.) and atmospheric pressure (1 atm).

Preferably, the polyols according to the invention comprise two OH groups (diols) and 2 to 6 carbon atoms, and are linear and saturated.

Preferentially, they may be chosen from propylene glycol (propane-1,2-diol), propane-1,3-diol, butylene glycol (butane-1,3-diol), pentylene glycol (pentane-1,2-diol), and mixtures thereof. Very preferentially, the polyol is propylene glycol.

The composition according to the invention preferably comprises said polyol(s) in a total amount ranging from 10% to 70% by weight, better still from 20% to 60% by weight, or even from 25% to 55% by weight, preferentially from 30% to 50% by weight and even better still from 35% to 45% by weight, relative to the total weight of the composition.

In a preferred embodiment, the composition according to the invention comprises one or more saturated linear C2-C6 diols in a total amount ranging from 10% to 70% by weight, better still from 20% to 60% by weight, or even from 25% to 55% by weight, preferentially from 30% to 50% by weight, and even better still from 35% to 45% by weight, relative to the total weight of the composition.

Preferentially, the composition according to the invention comprises propylene glycol in a total amount ranging from 10% to 70% by weight, better still from 20% to 60% by weight, or even from 25% to 55% by weight, preferentially from 30% to 50% by weight, and even better still from 35 to 45% by weight, relative to the total weight of the composition.

### 2/ Monoalcools

The composition according to the invention comprises one or more monoalcohols comprising from 2 to 6 carbon atoms.

Preferably, said monoalcohol(s) are linear and saturated. They may comprise from 2 to 4 carbon atoms. They do not comprise any oxyalkylene or glycerol-based groups.

Preferably, the monoalcohol is chosen from ethanol, propanol, isopropanol and butanol, and mixtures thereof, preferentially ethanol.

The composition according to the invention preferably comprises said C2-C6 monoalcohol(s) in a total amount ranging from 10% to 70% by weight, better still from 20% to 60% by weight or even from 25% to 55% by weight, relative to the total weight of the composition.

The composition according to the invention preferentially comprises ethanol in a total amount ranging from 10% to 70% by weight, better still from 20% to 60% by weight, or even from 25% to 55% by weight, relative to the total weight of the composition.

Preferably, the weight ratio between the total amount of polyols (i) and the total amount of C2-C6 monoalcohols (ii) is advantageously between 0.5 and 2, better still between 0.7 and 1.5.

Preferably, the total amount of polyol(s) (i) and of C2-C6 monoalcohols (ii) ranges from 30% to 99% by weight, preferentially from 50% to 98% by weight and better still from 60% to 95% by weight relative to the total weight of the composition.

### 3/ Fatty Amine

The composition according to the invention comprises one or more fatty amines.

The term “fatty amine” means a compound comprising at least one optionally (poly)oxyalkylenated primary, secondary or tertiary amine function, or salts thereof and comprising at least one C₆-C₃₀ hydrocarbon-based chain.

Preferably, the fatty amines according to the invention are not (poly)oxyalkylenated.

The fatty amines that may be used in the context of the invention may be fatty amidoamines, tertiary fatty amines or a mixture of these compounds.

Fatty amines that may thus be mentioned include fatty amidoamines, and notably fatty amidoamines in which the C₆-C₃₀ fatty chain may be borne by the amine group or by the amido group.

The term “amidoamine” means a compound comprising at least one amide function and at least one primary, secondary or tertiary amine function.

The term “fatty amidoamine” means an amidoamine comprising at least one C₆-C₃₀ hydrocarbon-based chain.

Preferably, the fatty amidoamines according to the invention are not quaternized.

Preferably, the fatty amidoamines according to the invention are not (poly)oxyalkylenated.

Among the amidoamines that may be used in the context of the invention, mention may be made of the amidoamines of formula A: RCONHR″N(R′)₂ (A)

in which:
R represents a monovalent hydrocarbon-based radical containing from 5 to 29 carbon atoms, preferably from 7 to 23 carbon atoms, and in particular a linear or branched, saturated or unsaturated C₅-C₂₉ and preferably a C₇-C₂₃ alkyl radical, optionally substituted with one or more hydroxyl (OH) groups; preferably a linear or branched C₅-C₂₉ and better still C₇-C₂₃ alkyl radical, or a C₅-C₂₉ and better still C₇-C₂₃ alkenyl radical; preferentially a linear or branched C₇-C₂₃ radical, which is saturated or comprising a double unsaturation (C═C);
R″ represents a divalent hydrocarbon-based radical, which is preferably linear, containing 1 to 6 carbon atoms, preferably 2 to 4 carbon atoms and better still 3 carbon atoms; and
R′, which may be identical or different, represent a linear or branched, saturated or unsaturated monovalent hydrocarbon-based radical containing 1 to 6 carbon atoms, preferably from 1 to 4 carbon atoms, preferably a methyl or ethyl radical.

Preferably, in formula (A):
R′ are identical and represent methyl;
R″ represents a linear divalent hydrocarbon-based radical containing 2 to 4 carbon atoms and better still 3 carbon atoms; and
R represents a C7-C23 linear or branched hydrocarbon-based radical, which is saturated or comprising an unsaturation (C═C).

The amidoamines of formula (A) may be chosen, alone or as a mixture, from the following compounds:
oleamidopropyldimethylamine,
stearamidopropyldimethylamine, notably the product sold by the company Inolex Chemical Company under the name Lexamine S13,
isostearamidopropyldimethylamine,
stearamidoethyldimethylamine,
lauramidopropyldimethylamine,
myristamidopropyldimethylamine,
behenamidopropyldimethylamine,
dilinoleamidopropyldimethylamine,
palmitamidopropyldimethylamine,
ricinoleamindopropyldimethylamine,
soyamidopropyldimethylamine,
avocadoamidopropyldimethylamine,
cocamidopropyldimethylamine,
minkamidopropyldimethylamine,
oatamidopropyldimethylamine,
sesamidopropyldimethylamine,
tallamidopropyldimethylamine,
olivamidopropyldimethylamine,
palmitamidopropyldimethylamine,
stearamidoethyldiethylamine,
brassicamidopropyldimethylamine.

Preferably, the fatty amidoamines are chosen from oleamidopropyldimethylamine, stearamidopropyldimethylamine, brassicamidopropyldimethylamine and mixtures thereof.

Fatty amines that may also be mentioned include tertiary fatty amines, and in particular the tertiary fatty amines of formula (B): RN(R′)₂

in which:
R represents a monovalent hydrocarbon-based radical containing from 6 to 30 carbon atoms, preferably from 8 to 24 carbon atoms, and in particular a linear or branched, saturated or unsaturated C₆-C₃₀ and preferably C₈-C₂₄ alkyl radical; preferably a linear or branched C₆-C₃₀ and better still C₈-C₂₄ alkyl radical; or a linear or branched C₆-C₃₀ and preferably C₈-C₂₄ alkenyl radical; and
R′, which may be identical or different, represent a linear or branched, saturated or unsaturated monovalent hydrocarbon-based radical containing 1 to 6 carbon atoms, preferably from 1 to 4 carbon atoms, preferably a methyl radical.

As fatty amines corresponding to formula B, mention may be made of the following compounds: dimethyllauramine, dimethylbehenamine, dimethylcocamine, dimethylmyristamine, dimethylpalmitamine, dimethylstearamine, dimethyltallowamine, dimethylsoyamine and mixtures thereof.

Preferably, the composition according to the invention comprises one or more fatty amines chosen from fatty amidoamines, notably corresponding to formula (A) as defined above, and more particularly chosen from oleamidopropyldimethylamine, stearamidopropyldimethylamine, brassicamidopropyldimethylamine and mixtures thereof.

The composition according to the invention preferably comprises said fatty amine(s) in a total amount ranging from 0.1% to 15% by weight, better still from 0.5% to 10% by weight or even from 1% to 5% by weight, relative to the total weight of the composition.

In a preferred embodiment, the composition according to the invention comprises said fatty amidoamines, notably of formula (A) as defined above, in a total amount ranging from 0.1% to 15% by weight, better still from 0.5% to 10% by weight or even from 1% to 5% by weight, relative to the total weight of the composition.

Better still, the composition according to the invention comprises fatty amidoamines chosen from oleamidopropyldimethylamine, stearamidopropyldimethylamine, brassicamidopropyldimethylamine and mixtures thereof, in a total amount ranging from 0.1% to 15% by weight, better still from 0.5% to 10% by weight or even from 1% to 5% by weight, relative to the total weight of the composition.

### 4/ Carboxylic Acid

The composition according to the invention comprises one or more carboxylic acids corresponding to formula (I) below:

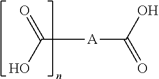

in which:
n is an integer between 0 and 10, better still between 1 and 5, even better still n =1 or 2, preferably n=2;
A is a saturated or unsaturated, linear, branched, cyclic or even aromatic monovalent hydrocarbon-based group (when n=0) or multivalent hydrocarbon-based group (when n is other than 0), comprising from 1 to 30 carbon atoms, preferably from 1 to 12 carbon atoms, better still from 1 to 6 carbon atoms, optionally substituted with one or more hydroxyl groups (OH) and/or amino groups (NR1R2 with R1 and R2, independently of each other, representing H or a C1-C4 alkyl).

Preferably, A is a monovalent or multivalent (C₁-C₆)alkylene or phenylene group, optionally substituted with one or more hydroxyl and/or amino groups.

Preferably, the carboxylic acids of formula (I) are alpha-hydroxy acids, for which A is a (C₁-C₆)alkylene and better still (C₂-C₄)alkylene group, or a phenylene group, substituted with 1 or 2 hydroxyl and/or amino groups, preferably 1 hydroxyl group; and n=0 to 2.

Mention may be made in particular of the carboxylic acids of formula (I) in which:
n=0 and A is a monovalent (C₁-C₆)alkyl and notably (C₂-C₄)alkyl group, optionally substituted with one or more hydroxyl groups (OH), notably 1 or 2 OH, preferably 1 OH;
n 32 0 and A is a phenyl radical substituted with 1 OH radical; or
n=1 or 2 and A is a divalent or trivalent (C₁-C₆)alkyl and better still (C₂-C₄)alkyl group, substituted with one or more hydroxyl and/or amino groups, notably 1 or 2 OH, preferably 1 OH.

Preferentially, the carboxylic acids may be chosen from:
citric acid (n=2 and A trivalent=—CH2—CHOH—CH2—)
salicylic acid (n=0 and A=phenyl substituted with an OH)
lactic acid (n=0 and A monovalent=—CH(OH)CH3)
tartaric acid (n=1 and A divalent=—CH(OH)—CH(OH)—) and
glutamic acid (n=1 and A divalent=—CH(NH2)—CH2—CH2—)

Even more preferentially, the carboxylic acid is citric acid.

The composition according to the invention preferably comprises said carboxylic acid(s) in a total amount of greater than or equal to 0.1% by weight, better still greater than or equal to 0.2% by weight, even better still greater than or equal to 0.3% by weight and preferentially greater than or equal to 0.4% by weight, relative to the total weight of the composition.

The composition according to the invention preferably comprises said carboxylic acid(s) in a total amount ranging from 0.1% to 5% by weight, better still ranging from 0.2% to 2.5% by weight, even better still from 0.3% to 1.5% by weight and preferentially from 0.4% to 1% by weight, relative to the total weight of the composition.

In a preferred embodiment, the composition according to the invention comprises citric acid in a total amount ranging from 0.1% to 5% by weight, better still from 0.2% to 2.5% by weight, even better still from 0.3% to 1.5% by weight and preferentially from 0.4% to 1% by weight, relative to the total weight of the composition.

### 5/ Fatty Alcohol Which is Preferably Solid

The composition according to the invention may advantageously comprise one or more fatty alcohols, preferably one or more solid fatty alcohols.

The solid fatty alcohols that may be used are solid at room temperature (25° C.) and at atmospheric pressure (1 atm.) and are water-insoluble, i.e. they have a solubility in water of less than 1% by weight and preferably less than 0.5% by weight, at 25° C., 1 atm.

The term “fatty alcohol” means an aliphatic alcohol comprising from 8 to 40 carbon atoms and comprising at least one hydroxyl group OH. These fatty alcohols are neither oxyalkylenated nor glycerolated.

The fatty alcohols are different from the polyols and the monoalcohols described above.

Preferably, the solid fatty alcohols have the structure R—OH with R denoting a linear alkyl group, optionally substituted with one or more hydroxyl groups, comprising from 8 to 40, better still from 10 to 30 or even from 12 to 24 carbon atoms.

Preferentially, the solid fatty alcohols have the structure R—OH with R denoting a linear alkyl group (not substituted with one or more OH groups), comprising from 8 to 40, better still from 10 to 30 or even from 12 to 24 carbon atoms.

The solid fatty alcohols that may be used may be chosen, alone or as a mixture, from:
lauryl alcohol (1-dodecanol);
myristyl alcohol (1-tetradecanol);
cetyl alcohol (1-hexadecanol);
stearyl alcohol (1-octadecanol);
arachidyl alcohol (1-eicosanol);
behenyl alcohol (1-docosanol);
lignoceryl alcohol (1-tetracosanol);
ceryl alcohol (1-hexacosanol);
montanyl alcohol (1-octacosanol);
myricyl alcohol (1-triacontanol).

Preferentially, the solid fatty alcohol is chosen from myristyl alcohol, cetyl alcohol, stearyl alcohol, behenyl alcohol and mixtures thereof such as cetylstearyl alcohol or cetearyl alcohol.

The composition according to the invention preferably comprises said fatty alcohol(s) in a total amount ranging from 0.5% to 20% by weight and preferentially from 1% to 18% by weight relative to the total weight of the composition.

Better still, the composition according to the invention preferably comprises said solid fatty alcohol(s) in a total amount ranging from 0.5% to 20% by weight and preferentially from 1% to 18% by weight relative to the total weight of the composition.

### 6/ Other Ingredients

The composition according to the invention may advantageously also comprise one or more fatty substances other than the above fatty alcohols, and which are advantageously liquid, better still chosen from triglycerides of plant or synthetic origin, in particular plant oils, and esters of fatty acids and/or of fatty alcohols, and also mixtures thereof.

Among the triglycerides of plant or synthetic origin, mention may be made of liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, jojoba oil, shea butter oil and caprylic/capric acid triglycerides, for instance those sold by the company Stéarinerie Dubois or those sold under the names Miglyol® 810, 812 and 818 by the company Dynamit Nobel.

As regards the fatty acid and/or fatty alcohol esters, which are advantageously different from the triglycerides mentioned above, mention may notably be made of esters of saturated or unsaturated, linear or branched C₁-C₂₆ aliphatic mono- or polyacids and of saturated or unsaturated, linear or branched C₁-C₂₆ aliphatic monoor polyalcohols, the total carbon number of the esters more particularly being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononanoate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate. Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

Preferably, the liquid fatty substance(s) other than the fatty alcohols are chosen from triglycerides of plant or synthetic origin.

The composition according to the invention preferably comprises said fatty substance(s) other than the fatty alcohols in a total amount ranging from 0.1% A to 15% by weight and preferentially from 0.5% to 10% by weight relative to the total weight of the composition.

Preferentially, the composition according to the invention comprises said liquid fatty substance(s) other than the fatty alcohols, and notably said triglyceride(s) of plant or synthetic origin, in a total amount ranging from 0.1% to 15% by weight, preferentially from 0.5% to 10% by weight, relative to the total weight of the composition.

A subject of the present invention is also a cosmetic process for treating keratin fibers, in particular human keratin fibers such as the hair, comprising the application to said keratin fibers of a composition as defined previously.

The cosmetic composition according to the invention may be applied to dry or wet keratin fibers, preferably wet keratin fibers, that have optionally been washed with shampoo beforehand.

Advantageously, the composition is left on the keratin fibers for a leave-on time ranging from 1 minute to 20 minutes.

The keratin fibers are preferably rinsed after application of said cosmetic composition, for example with water.

The process according to the invention is in particular a hair treatment process, which makes it possible, for example, to provide care and/or conditioning to keratin fibers, in particular to the hair, notably disentangling, smoothness and/or suppleness.

The examples that follow serve to illustrate the invention without, however, being limiting in nature.

In the examples that follow, unless otherwise indicated, the amounts are indicated as weight percentages of active material (% AM) relative to the total weight of the composition.

### EXAMPLE 1

Compositions A and B according to the invention are prepared from the ingredients whose contents are indicated in the table below (g% AM):

TABLE 1

| | Composition A | Composition B |
|---|---|---|
| Stearamidopropyldimethylamine | 3 | 3 |
| Citric acid | 0.51 | 0.51 |
| Caprylic/capric triglyceride | 0.9 | 9.8 |
| Cetearyl alcohol | — | 12.5 |
| Myristyl alcohol | 2 | 3.4 |
| Ethanol | 50 | 30 |
| Propylene glycol | qs 100 | qs 100 |

Transparent liquid compositions are obtained, which are easy to apply to moistened or wet hair, which has preferably been washed beforehand; they may be used as rinse-out hair conditioners.

After application of the composition and rinsing with water, it is found that the hair is more supple and has a smooth feel; it is also shiny and easy to disentangle.

1-16. canceled

17. An anhydrous cosmetic composition comprising:
(i) one or more polyols;
(ii) one or more monoalcohols comprising from 2 to 6 carbon atoms;
(iii) one or more fatty amines; and
(iv) at least 0.1%, by weight, relative to the total weight of the composition, of one or more carboxylic acids comprising from 1 to 6 carbon atoms.

18. The anhydrous cosmetic composition according to claim 17, comprising (i) one or more polyols chosen from propylene glycol (propane-1,2-diol), propane-1,3-diol, butylene glycol (butane-1,3-diol), pentylene glycol (pentane-1,2-diol), or mixtures of two or more thereof.

19. The anhydrous cosmetic composition according to claim 17, wherein the total amount of polyols ranges from 10% to 70% by weight, relative to the total weight of the composition.

20. The anhydrous cosmetic composition according to claim 17, comprising (ii) one or more monoalcohols chosen from linear and saturated monoalcohols comprising from 2 to 6 carbon atoms.

21. The anhydrous cosmetic composition according to claim 17, wherein the total amount of monoalcohols ranges from 10% to 70% by weight, relative to the total weight of the composition.

22. The anhydrous cosmetic composition according to claim 17, having a weight ratio of the total amount of (i) polyols to the total amount of (ii) monoalcohols comprising from 2 to 6 carbon atoms ranging from 0.5 to 2.

23. The anhydrous cosmetic composition according to claim 17, comprising (iii) one or more fatty amines chosen from fatty amidoamines, tertiary fatty amines, or mixtures thereof.

24. The anhydrous cosmetic composition according to claim 17, comprising one or more fatty amines chosen from fatty amidoamines of formula (A):
RCONHR″N(R′)₂ (A)
wherein:
R is a monovalent hydrocarbon-based radical comprising from 5 to 29 carbon atoms, optionally substituted with one or more hydroxyl (OH) groups;
R″ is a divalent hydrocarbon-based radical comprising from 1 to 6 carbon atoms; and
R′, which may be identical or different, is a linear or branched, saturated or unsaturated, monovalent hydrocarbon-based radical comprising from 1 to 6 carbon atoms.

25. The anhydrous cosmetic composition according to claim 17, comprising one or more fatty amines chosen from tertiary fatty amines of formula (B):
RN(R′)₂ (B)
wherein:
R is a monovalent hydrocarbon-based radical comprising from 6 to 30 carbon atoms; and
R′, which may be identical or different, is a linear or branched, saturated or unsaturated monovalent hydrocarbon-based radical comprising from 1 to 6 carbon atoms.

26. The anhydrous cosmetic composition according to claim 17, wherein the total amount of fatty amines ranges from 0.1% to 15% by weight, relative to the total weight of the composition.

27. The anhydrous cosmetic composition according to claim 17, comprising (iv) one or more carboxylic acids chosen from carboxylic acids of formula (I): 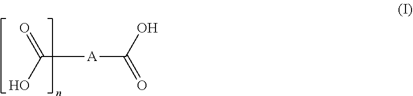
wherein:
n is an integer ranging from 0 to 10; and
A is chosen from:
a saturated or unsaturated, linear, branched, cyclic or aromatic monovalent hydrocarbon-based group when n is 0, or
a multivalent hydrocarbon-based group comprising from 1 to 30 carbon atoms, optionally substituted with one or more hydroxyl groups and/or amino groups when n is other than 0.

28. The anhydrous cosmetic composition according to claim 17, wherein the total amount of carboxylic acids ranges from 0.1% to 5% by weight, relative to the total weight of the composition.

29. The anhydrous cosmetic composition according to claim 17, further comprising one or more fatty alcohols of the formula:
R—OH
wherein R is a linear alkyl group, optionally substituted with one or more hydroxyl groups, comprising from 8 to 40 carbon atoms.

30. The anhydrous cosmetic composition according to claim 29, comprising one or more fatty alcohols chosen from lauryl alcohol (1-dodecanol), myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricyl alcohol (1-triacontanol), or mixtures of two or more thereof.

31. The anhydrous cosmetic composition according to claim 29, wherein the total amount of fatty alcohols ranges from 0.5% to 20% by weight, relative to the total weight of the composition.

32. The anhydrous cosmetic composition according to claim 17, further comprising one or more liquid fatty substances chosen from triglycerides of plant or synthetic origin.

33. The anhydrous cosmetic composition according to claim 32, wherein the total amount of liquid fatty substances ranges from 0.1% to 15% by weight, relative to the total weight of the composition.

34. The anhydrous cosmetic composition according to claim 17, wherein the combined total amount of (i) polyols and (ii) monoalcohols comprising from 2 to 6 carbon atoms ranges from 30% to 99% by weight, relative to the total weight of the composition.

35. The anhydrous cosmetic composition according to claim 17, comprising less than 0.5% water by weight, relative to the total weight of the composition.

36. A method for treating keratin fibers, the method comprising applying to the keratin fibers an anhydrous cosmetic composition comprising:
(i) one or more polyols;
(ii) one or more monoalcohols comprising from 2 to 6 carbon atoms;
(iii) one or more fatty amines; and
(iv) at least 0.1%, by weight, relative to the total weight of the composition, of one or more carboxylic acids comprising from 1 to 6 carbon atoms.
